(19) United States
(12) Patent Application Publication
(10) Pub. No.: US 2009/0311328 A1
(43) Pub. Date: Dec. 17, 2009

(54) Bulking of Soft Tissue

(75) Inventors: Paul Wilhelm Richter, Wingate Park (ZA); Kersch Naidoo, Port Shepstone (ZA); Schalk Willen Van Der Merwe, Pretoria (ZA)

Correspondence Address: THE NATH LAW GROUP, 112 South West Street, Alexandria, VA, 22314, US

(73) Assignee: CSIR, Pretoria (ZA)

(21) Appl. No.: 12/225,707

(22) PCT Filed: Mar. 30, 2007
(86) PCT No.: PCT/IB2007/051153

§ 371 Date: Sep. 29, 2008

(30) Foreign Application Priority Data
Mar. 31, 2006 (ZA) 2006/02676

(51) Int. Cl. A61K 9/14

(52) U.S. Cl. 424/489

(57) ABSTRACT

A soft tissue bulking material includes a plurality of particles. Each particle comprises a rounded polymeric shell defining an internal cavity and having a maximum outer dimension of 50 μm-250 μm. A port or opening is provided in the shell. The port or opening thus provides access to the cavity. The port or opening has a size or dimension that ranges from one tenth of the particle's outer dimension up to the particle's outer dimension.

THIS INVENTION relates to the bulking of soft tissue. It relates in particular to a soft tissue bulking material, and to an injectable soft tissue bulking composition.

According to a first aspect of the Invention, there is provided a soft tissue bulking material, which includes a plurality of particles, with each particle comprising a rounded polymeric shell defining an internal cavity and having a maximum outer dimension of 50 μm-250 μm, and a port or opening in the shell, with the port or opening thus providing access to the cavity, and with the port or opening having a size or dimension that ranges from one tenth of the particle's outer dimension up to the particle's outer dimension.

The bulking material of the invention is suitable for use as a soft tissue bulking material in the treatment of gastroesophageal reflux disease (GERD), urinary reflux disease, stress urinary incontinence (SUI), faecal incontinence, augmentation of dermal irregularities, vocal fold augmentation for the treatment of vocal fold paralysis, or the like. It is applied by injecting it, when suspended in a carrier medium as hereinafter described, into soft tissue requiring bulking or augmentation.

The internal cavity is thus enclosed by the shell, with the port or opening providing external access to the cavity.

The shells of the particles are preferably substantially spherical so that their outer diameters are thus 50 μm-250 μm. In other words, the particles are preferably hollow microspheres each having a single dominant large port or opening in its shell. The diameters of the microspheres may typically be about 100 μm.

The ports of the particles may then be substantially circular with their sizes or diameters thus ranging from one tenth of the diameters of the particles up to the respective diameters of the particles. The diameters of the ports may be in the range of 20 μm to 100 μm. For example, the port diameters may be about 60 μm.

The shells of at least some of the particles may have micropores. The micropores may have dimensions <10 μm, eg diameters <10 μm.

The shells of at least some of the particles may have macropores. The macropores may have dimensions, eg diameters, of from 10 μm to 50 μm.

In one embodiment of the invention, less than 20% of the outer surface areas of the shells, that is, excluding the internal surface areas of any micro- or macropores present, of the particles may be occupied by macropores i.e. the microspheres may have limited macroporosity.

The shell may include, as a composite with the polymer or absorbed onto the polymer (or otherwise attached thereto), at least one additive selected from a calcium phosphate compound, a contrast agent, a therapeutic agent, a growth factor, autologous platelet rich plasma, normal human cells, and autologous stem cells.

According to a second aspect of the invention, there is provided an injectable soft tissue bulking composition, which includes
(i) a soft tissue bulking material as hereinbefore described; and
(ii) a compatible carrier medium in which the bulking material particles are suspended, with the composition having a consistency that renders it suitable for application by injecting it into soft tissue.

The carrier medium may include a carrier medium material selected from collagen, chitosan, alginate, polyvinyl pyrrolidone, silicone oil, gelatin, fat, hyaluronic acid, saline, water, plasma, aqueous solution, glycols, medium chain triglycerides, glycerides, glycerol, B-glucan & agarose solution, ethyl lactate, hydroxypropyl methylcellulose, poloxamers or poly (N-isopropylacrylamide) or a derivative thereof, dissolved in a solvent.

The ratio of carrier medium material to solvent may be from 1:1 to 200:1 (mg of carrier medium to ml of solvent).

Any suitable solvent may be used, such as water, an acid or a base, depending on the carrier medium material utilised. Thus, when collagen is used as the carrier medium, the solvent may be acetic acid. When alginate is used as the carrier medium, a base such as sodium hydroxide can be used as the solvent.

The carrier medium may be a pseudoplastic liquid, such as hyaluronic acid (preferably derived from a non-animal source) dissolved in water, that allows for a reduced viscosity under high shear such as when injecting it, but has higher viscosity for stabilizing or suspending the injected material after injection. Instead, the carrier medium may be liquid at room temperature, and may be adapted to undergo a phase change, e.g. from liquid to a gel, when injected into soft tissue i.e. at human body conditions. In particular, the carrier medium may be temperature and/or pH responsive so that at body temperature and/or body pH, i.e. after injection into human soft tissue, it undergoes a phase change from liquid to gel. Preferably, highly purified glutaraldehyde crosslinked bovine collagen is used as the carrier medium material. An example thereof is a collagen preparation manufactured by INAMED Aesthetics of Santa Barbara, Calif., USA and marketed by C. R. Bard of Murray Hill, N.J., USA.

The composition is formed by combining the bulking material and the carrier medium. The bulking material is preferably sterilized before adding it to the carrier medium. This may be effected by gamma-sterilizing it to a dose of 25 kGy. The carrier medium may also be sterilized before the bulking material is added thereto. Aqueous collagen solutions can be sterilized either by filtration with in-line sterile filters (0.22 micrometer) and through strictly sterile preparation procedures.

The carrier medium may include an additive as hereinbefore described, such as a contrast agent, autologous platelet rich plasma, normal human cells, or autologous stem cells.

The soft tissue bulking material may be prepared by
(i) dispersing a pore forming agent in a solution of a polymer dissolved in a solvent, to form an oil (O) phase;
(ii) adding the oil phase to a water (W) phase comprising an emulsifying agent/surfactant dissolved in water, and forming an oil-in-water emulsion (O/W); or
(iii) adding a water phase (W) comprising an emulsifying agent/surfactant dissolved in water, to the oil phase, and forming an emulsion, thereafter adding this emulsion to a second oil phase containing an emulsifying agent/surfactant, and forming a water-in-oil-in-oil emulsion ((W/O)/O);
(iv) if appropriate, adding an acid to the emulsion of step (ii) or (iii), with the acid reacting with the pore forming agent, thereby forming rounded polymeric shells each defining an internal cavity and having a maximum outer dimension of 50 μm-250 μm, and a port or opening in the shell, with the port or opening thus providing access to the cavity, and with the port of cavity having a maximum dimension of 100 μm and a minimum dimension of 20 μm.

Instead, the soft tissue bulking material may be prepared by
(v) adding a water phase (W) comprising an emulsifying agent/surfactant dissolved in water, to an oil phase (O) comprising a solution of a polymer dissolved in a solvent, and forming a (W/O) emulsion; adding a pore forming agent to this emulsion; thereafter
(vi) adding this emulsion back into a water phase (W) comprising an emulsifying agent/surfactant dissolved in water, and forming a water-in-oil-in-water emulsion ((W/O)/W), and then
(vii) if appropriate, adding an acid to the ((W/O)/W) emulsion, with the acid reacting with the pore forming agent, thereby forming rounded polymeric shells each defining an internal cavity and having a maximum outer dimension of 50 μm-250 μm, and a port or opening in the shell, with the port or opening thus providing access to the cavity, and with the port of cavity having a maximum dimension of 100 μm and a minimum dimension of 20 μm.

The pore forming agent and acid can thus be selected so that, when the acid is added to the emulsion, it reacts with the pore forming agent resulting in effervescence or foaming taking place, which leads to the formation of the ports in the particles.

The pore forming agent may be a solid pore forming agent or porogen, which may be selected from calcium carbonate, sodium carbonate, sodium bicarbonate, ammonium carbonate, ammonium bicarbonate, a nitrate that is acceptable for in vivo use, sodium chloride, sodium citrate, saccharose and glucose. Typically, sodium bicarbonate is used. However, instead, an inert liquid pore forming agent, such as a perfluorocarbon, may be used; however, the porogen utilized is not limited to the above-mentioned substances

It will be appreciated that the step of adding the acid to the emulsion will only be used when appropriate, i.e. when the solid or volatile organic solvent pore forming agent or porogen by itself does not generate enough gas When a chemically inert liquid pore forming agent is used, there will be no acid addition. Furthermore, the acid addition can also be dispensed with when certain solid pore forming agents are used. For example, when ammonium bicarbonate is used as the porogen, no acid addition is required since ammonium bicarbonate is sufficiently reactive for pore forming to occur without an acid having to be present.

The mass ratio of polymer to pore forming agent may be from 1:5 to 2:1, typically about 1:2.

When a solid pore forming agent or porogen is used, it may have a particle size range of 0.01 μm-250 μm, typically about 150 μm.

After the pore forming agent has been added to the solution, the porogen solution mixture is preferably stirred or homogenized until a homogeneous dispersion is achieved.

The ratio of polymer to solvent in the solution may be from 1:20 to 1:5 (gms polymer to ml of solvent), typically about 1:6 depending on the polymer solubility limits.

The polymer may be a synthetic polymer selected from poly(epsilon-caprolactone), polylactide, polyglycolide, polylactide-co-glycolide, poly(epsilon-caprolactone)-co-glycolide, polyhydroxybutyrate, polyhydroxyvalerate, polybutyrolactone, polyvalerolactone, poly(ethylene carbonate), poly(ethylene terephthalate), polydioxanone, polyurethane, polyethylene glycol, polymethylmethacrylate, polyvinyl acetate and poly (2-hydroxyethyl methacrylate) or a natural polymer selected from collagen, hyaluronic acid, chitosan, fibrin and alginate.

The solution is thus formed by dissolving the polymer in the solvent, e.g. using stirring or homogenization. The solvent may be pre-saturated with some of the water phase used in step (ii), (iii) or (v).

The solvent may be a water-soluble organic solvent such as an aromatic hydrocarbon, a chlorinated solvent (dichloromethane, chloroform, CCl4 or the like), an alcohol (benzyl alcohol, polypropylene glycol, n-butanol, or the like), an ester (ethyl acetate, butyl acetate, methyl benzoate, methyl acetate, or the like), or an organic acid such as acetic acid or propionic acid.

The water phase may comprise emulsifying agent/surfactant to deionised water in a ratio of 1:10 to 1:1000 (gms emulsifying agent/surfactant to ml of water), typically about 1:50.

In steps (v) and (vi) above, the make-up or composition of the water phases used may be the same.

The emulsifying agent/surfactant may be selected from polyvinyl alcohol, gelatin, polyethylene glycol, sodium dodecylsulfate, polysorbate, polyvinylpyrrolidone, poloxamers, glyceryl monooleate, glyceryl monostearate, polyoxyethylene alkyl ether, hydroxypropyl cellulose, hydroxypropylmethyl cellulose, and mixtures thereof.

The water phase is formed by stirring or homogenizing the deionized water after the emulsifying agent/surfactant has been added thereto, until the emulsifying agent/surfactant has dissolved. The deionized water may be pre-saturated with the solvent used in the solution of step (i) above.

The second oil phase thus comprises an oil and the emulsifying agent/surfactant. The oil of the second oil phase is different to the oil, ie the solvent, of the other or first oil phase. The oil of the second oil phase may be vegetable oil, mineral oil, jojoba oil, avocado oil or palm kernel oil. The volumetric proportion of oil to emulsifying agent/surfactant in the second oil phase may be from 20:1 to 2000:1, typically about 200:1. The emulsion to which the acid is added is thus an O/W emulsion, or a (W/O)/W emulsion, or a (W/O)/O emulsion.

The O/W emulsion is formed by adding the oil phase (O) to the water phase (W) while continuing to stir or homogenize the W phase. The ratio of W phase to O phase may be 5:1 to 200:1 (ml of W phase to ml of O phase), typically about 20:1.

The (W/O)/W emulsion is formed by adding the W phase (the initial W phase) to the O phase, and forming an emulsion, and then adding this emulsion back into a W phase, thereby forming the (W/O)/W emulsion.

The (W/O)/O emulsion is formed by adding the W phase to the O phase, and forming an emulsion, and then adding this emulsion to the second oil phase, thereby forming the (W/O)/O emulsion.

In all cases, the emulsification may be effected by magnetic stirring, membrane emulsification, rotor-stator homogenization, high pressure homogenization or ultrasonic homogenization. Stirring/homogenization is continued until complete emulsification has been achieved. It can also be appreciated that other emulsification methods known to those in the art such as spray drying can instead be used to produce the desired microparticles.

The acid addition may be effected while emulsifying to stir or homogenize the emulsion. Sufficient acid is used to balance stoichiometrically the amount of porogen used. The acid may be selected from acetic acid, ascorbic acid, salicylic acid, phosphoric acid, hydrochloric acid, propionic acid and mixtures thereof.

After the acid addition to the emulsion, the resultant mixture may be solvent evaporated under vacuum at 50-1000 mbar (abs) to permit formation of the shells and ports therein. Typically, the solvent evaporation may be effected at a vacuum of about 500 mbar (abs), until the acid/porogen reaction has been completed.

A contrast agent, which can be either water soluble or water insoluble, can be added to the O phase so that it is incorporated into the polymeric shells and/or added to the carrier medium. Examples of water soluble contrast agents include metrizamide, lopamidol, iothalamate sodium, iodomide sodium, and meglumine. Water insoluble contrast agents include tantalum, tantalum oxide, gold, tungsten, platinum, perfluorocarbons and barium. These additives enable a person injecting the composition to visualize, by means of fluoroscopic, radiographic, ultrasonic optical coherence tomography and/or other visual imaging equipment, the extent of soft tissue augmentation during injection, allowing a more controlled procedure.

A therapeutic agent such as anti-biotic or an anti-inflammatory may be added to the W phase for incorporation into the polymeric shells and/or added to the carrier medium.

A growth factor to stimulate the initial stages of new tissue formation and vascularization may be incorporated into the polymeric shells and/or into the carrier medium.

Growth factors that can be used Include heparin, epidermal growth factor, transforming growth factors, transforming growth factor-β platelet derived growth factor, fibroblast growth factor, connective tissue activating peptides, β-thromboglobulin, insulin-like growth factor, tumour necrosis factors, interleukins, colony stimulating factors, erythropoietin, nerve growth factors, interferons, osteogenic factors and bone morphogenetic proteins.

Autologous platelet rich plasma (PRP) can also be incorporated into the polymeric shells and/or into the carrier medium, if desired. Such an additive serves as a rich source of a cocktail of various relevant growth factors, and will stimulate the initial stages of new tissue formation and vascularization.

Relevant normal human cells and/or autologous stem cells to further stimulate new tissue formation and vascularization may also be incorporated into the polymeric shells and/or into the carrier medium. These may include adult or pre-differentiated adipose-derived stem cells, myoblasts, osteoblasts, fibroblasts, epithelial and endothelial cells, smooth muscle cells, preferably adult adipose-derived stem cells, and normal human smooth muscle and epithelial cells. The stem cells may be pre-differentiated in vitro.

The bulking composition of the Invention can thus be used to bulk soft tissue, by injecting the composition into soft tissue requiring bulking. The injection of the composition may be effected cytoscopically, endoscopically or laparoscopically.

As hereinbefore described, the soft tissue applications may include treatment of GERD, urinary reflux disease, stress urinary Incontinence (SUI), faecal incontinence, augmentation of dermal irregularities, and vocal fold augmentation for the treatment of vocal fold paralysis.

The invention will now be described in more detail with reference to the accompanying diagrammatic drawings in which

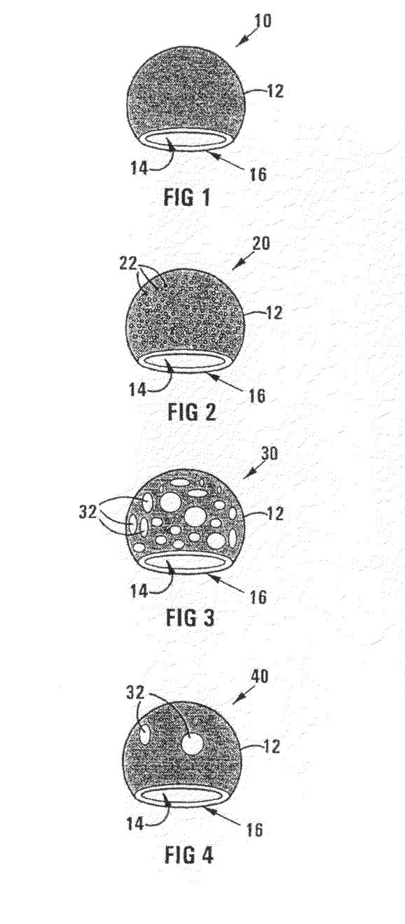

FIG. 1 shows a three-dimensional view of a particle of a soft tissue bulking material according to a first embodiment of the invention;

FIG. 2 shows a similar three-dimensional view of a soft tissue bulking material according to second embodiment of the invention;

FIG. 3 shows a three-dimensional view of a particle of a soft tissue bulking material according to a third embodiment of the invention;

FIG. 4 shows a three-dimensional view of a particle of a soft tissue bulking material according to a fourth embodiment of the invention;

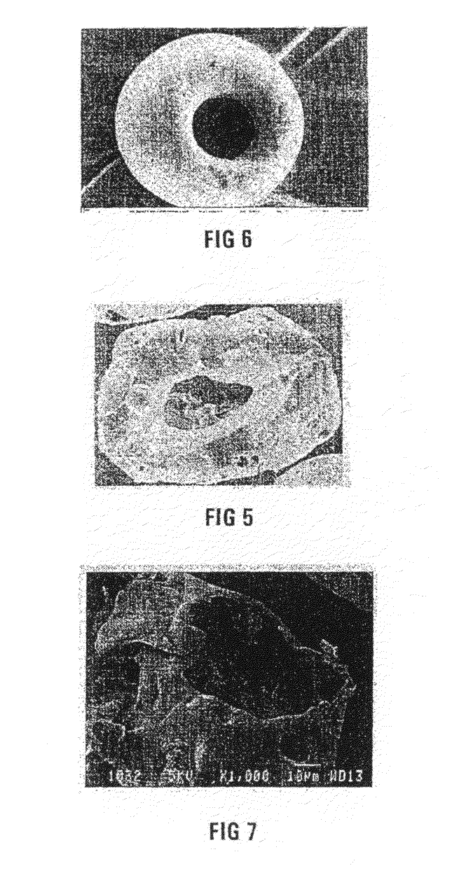

FIG. 5 is a scanning electron micrograph of a particle of the soft tissue bulking material as obtained from Example 1;

FIG. 6 is a scanning electron micrograph of a particle of the soft tissue bulking material as obtained from Example 2; and

FIG. 7 shows is a scanning electron micrograph of cells cultured on the microparticles as obtained from Example 1.

In the drawings, similar features are indicated with the same reference numerals.

Referring to FIG. 1, reference numeral 10 generally indicates a particle of a soft tissue bulking material according to a first embodiment of the invention.

The particle 10 includes spherical shell 12 of solid, I.e. non-porous polymer. The shell 12 typically has an outer diameter of about 100 μm. The polymer typically is poly(epsilon-caprolactone).

The spherical shell 12 defines a central enclosed rounded cavity 14.

The shell 12 typically has a wall thickness in the range of 1 □m to 10 □m.

A single dominant port, indicated by reference numeral 16, is provided In the shell 12. The diameter of the port is typically about 60 μm. The port 16 thus provides external access to the cavity 14.

Referring to FIG. 2, reference numeral 20 generally indicates a particle of a soft tissue bulking material according to a second embodiment of the invention.

In the case of the particle 20, the shell 12 is provided with micropores whose diameters are ≦10 μm.

Referring to FIG. 3, reference numeral 30 generally indicates a particle of a soft tissue bulking material according to a third embodiment of the invention.

The shell 12 of the particle 30 is provided with macropores 32 whose diameters are 10 to 50 μm.

Referring to FIG. 4, reference numeral 40 generally indicates a particle of a soft tissue bulking material according to a fourth embodiment of the invention.

The particle 40 has limited macro porosity i.e. the total open area of its macropores 32 is less than 20% of the total micro shell outer surface area.

The particles 10, 20, 30 and 40 are prepared or synthesized as hereinafter described.

The volume of injectable soft tissue bulking composition and soft tissue bulking material to be injected is approximately 0.5-20 ml, depending on the amount of soft tissue augmentation required. The polymeric microshells typically make up 10-20% of the injectable volume, thus approximately 0.5-4 ml endoscopically of polymeric microshells are required per treatment. The volume of polymeric microshells produced by the synthesis procedures described hereunder is approximately 1 ml.

### EXAMPLE 1

### O/W Procedure

Make up a 1% (w/v) polyvinyl alcohol (PVA) solution in 150 ml distilled water (W). Dissolve 1.5 g poly (epsilon-caprolactone) (PCL) in 10 ml dichloromethane to form an O phase. Add 3 g NaHCO₃ (particle size: 25-40 μm) acting as the porogen to the O phase. Gently magnetically stir oil-porogen mixture for 1 minute. Add oil phase to PVA solution (W phase) and homogenise for 2 minutes at 300 rpm. Then stir magnetically at 800 rpm at 20° C. for 2 hours until solvent evaporation has completed. Add 2.4 ml acetic acid after 1 hour of stirring. Allow foaming reaction to occur. Filter solution using an appropriate mesh size, to remove excess water and to obtain the desired particle size range, with a volume-derived yield of 80% of particles in the desired size range being achieved. The same filtration procedure is used in Examples 2 to 4 hereunder. Vacuum dry particles and separate using a typical coagulating procedure, to obtain a soft tissue bulking material in accordance with the invention. Volume averaged particle size as obtained by laser light scattering was 185 μm—see FIG. 5 which shows a scanning electron micrograph of a particle in accordance to the invention

### EXAMPLE 2

### W/0/W Procedure

Dissolve 1.5 g PCL in 10 ml dichloromethane (DCM) to form an oil (O) phase. Add 0.5 ml of a 1% (w/v) polyvinyl alcohol (PVA) solution (W) to the oil phase. Magnetically stir to form a first emulsion (W/O). Add 3 g CaCO₃ (particle size 100-150 μm) acting as the porogen to the first emulsion. Add the first emulsion to 150 ml of 1% PVA (w/v) (W/O/W). Stir this mixture magnetically at 800 rpm to form a second emulsion, and continue stirring till solvent evaporation has been completed. Add 2.4 ml acetic acid after 1 hour of stirring. Allow foaming reaction to occur. Rinse spheres three times with distilled water. Filter solution, and vacuum dry particles and separate using a typical coagulating procedure, to obtain the microshells i.e. soft tissue bulking material in accordance with the invention. Volume averaged particle size as obtained by laser light scattering was 196 μm—see FIG. 6 which shows a scanning electron micrograph of a particle in accordance to the invention

### EXAMPLE 3

### W/O/O Procedure

Make up a 0.33% (w/v) polyvinyl alcohol (PVA) solution in 5 ml distilled water (W). Dissolve 1 g poly(epsilon-caprolactone) (PCL) in 10 ml dichloromethane to form a first oil (O₁) phase. Add 3 g NaHCO3 (particle size 150-212 μm) acting as the porogen to the O₁ phase and gently stir magnetically the first emulsion (W/O₁) with porogen for 1 minute. A second oil phase (O₂) is made from 200 ml Vegetable Oil with 1% Span 60 (v/v). Span 60 (trademark) is a commercially available sorbitan monostearate, i.e. a surfactant/emulsifying agent. Add the first emulsion to the second oil phase (W/O₁/O₂) and stir magnetically at 2000 rpm for 2 hrs for emulsion formation and continue until solvent evaporation has completed. Filter the final emulsion and wash 3 times with de-ionised water. Remove residual oil on the formed microparticles with an appropriate solvent. Vacuum dry particles and separate using a typical coagulating procedure, to obtain the soft tissue bulking material in accordance with the invention.

### EXAMPLE 4

### (O/W) Procedure with a Liquid Porogen

Make up a 1% [w/v] polyvinyl alcohol (PVA) solution in 150 ml distilled water (W). Dissolve 1.5 g poly (epsilon-caprolactone) (PCL) in 10 ml dichloromethane to form an O phase. Add 3 g NaHCO₃ (particle size: 25-40 μm) to the O phase which acts as the solid porogen and 1 ml perfluorocarbon (PFC) which acts as the liquid porogen. Stir the oil-porogen-PFC mixture for 1 minute. Add oil phase to PVA solution (O/W) and homogenise for 2 minutes at 300 rpm. Then stir magnetically at 800 rpm at 20° C. for 2 hours until solvent evaporation has completed. Filter solution, vacuum dry particles and separate using a typical coagulating procedure, to obtain the soft tissue bulking material in accordance with the invention.

It will be appreciated that, when following the above procedures, the soft tissue bulking material will in each case comprise a mixture of the particles 10, 20, 30 and 40 of FIGS. 1, 2, 3 and 4 respectively.

### EXAMPLE 5

### In Vitro Smooth Muscle Cells Growth

To investigate the ability of smooth muscle cells to adhere on the soft tissue bulking material of Example 1, fresh smooth muscle cells were cultured in the presence of the above microparticles using the classical micro-carrier cell culturing technique. After a 120 hour period, most cells migrated Into the microparticles cavity which showed a preferential attachment to the polymer interior—see FIG. 7 which shows a scanning electron micrograph of cells cultured on the soft tissue bulking material in accordance with the invention

The embodiments of the present invention are intended to be merely exemplary and those skilled in the art will be able to ascertain numerous equivalents to the specific procedures used here. All such equivalents are considered to be within the scope of the present invention.

Preferably, highly purified glutaraldehyde crosslinked bovine collagen is used as the carrier medium material. The best known example of this is a collagen preparation manufactured by Collagen Corporation (Inamed Corporation) and marketed by C. R. Bard. Hyaluronic acid can also be used from Restylane™ (Q-Med) if a patient is found to be allergic to the bovine collagen.

Although the composition according to the invention, and in particular the bulking material, can be used to treat substantially any incontinence or reflux based diseases, it is believed that it will have particular application in the treatment of GERD, which arises primarily from the transient relaxation of the lower esophageal sphincter muscle (LES), which allows stomach acid to reflux up the esophagus. In other instances GERD can be attributed to the decreased resting tone of the LES. By endoscopic or laparoscopic injection of the bulking material into the lower esophageal sphincter muscle, it is augmented. The bulking arises from the particular configuration of the microspheres or shells of the particles of the bulking material, which permit tissue regeneration through tissue ingrowth into the cavities of the particles. In particular, it is believed that the single dominant large ports in the particles provide for enhanced or faster new tissue ingrowth without compromising mechanical integrity of the particles. Without wishing to be bound by theory, it is believed that the cavities in the particles and the dominant ports provide a stress free environment for new tissue formation, thus forming a ‘tissue harbour’ and giving cells a true three-dimensional environment in which to interact.

It is believed that providing the stress free environment or ‘tissue harbour’ for formation of new tissue would minimize fibrosis (scar tissue) and allow maximum volume of tissue formation, thereby improving muscle functioning. This should lead to a high rate of new tissue formation. This stress free environment should also be advantageous if the particles are precultured with suitable cells prior to implantation, as the cells located inside the particles will be protected from the high shear forces encountered on implantation, while having access to the surrounding tissue through the single large port, and to nutrients and oxygen supply through the port and through the micro- and macropores as weir, if these are present.

It is believed that by treating GERD with bulking material according to the invention, particularly good results will be achieved and problems associated with other methods of treating GERD will be avoided. For example, a patient will avoid having to take daily medication for treating GERD and major surgery can be avoided.

Furthermore, the particles biodegrade as time progresses, thereby promoting long-term tissue regeneration.

Effectively two bulking mechanisms come into play, namely: early stage bulking which arises from the initial volume of the Injected composition, and later bulking brought about by new tissue formation and possibly function restoration.

When the particles contain additives as hereinbefore described, still further advantages can arise. Thus, when the particles contain human cells or autologous stem cells (which could be predifferentiated into smooth muscle cells), this will further stimulate new tissue formation and vascularization, and can lead to muscle function being restored. When the particles contain relevant growth factors, these can accelerate early stages of new tissue formation. Additionally, such growth factors are released over a long period of time as the particles biodegrade. When the particles contain tricalcium phosphate, this provides mechanical strength and also acts as a calcium reservoir for supplying calcium to the body over a period of time. Tricalcium phosphate is a well-established bioactive material, and thus its incorporation into the bulking material particles boosts bioactivity of the particles, which leads to improved tissue ingrowth into the particles. Also, calcium participates in clotting cascades, and contributes to granulation of platelets.

Autologous platelet-rich plasma (PRP), which provides a rich source of a ‘cocktail’ of growth factors, is another possible beneficial additive. PRP will normally be incorporated into the carrier medium rather than into the particle shells, and thus can be released rapidly into the body since the carrier medium is rapidly resorbed. The PRP releases growth factors as the body sees fit, with calcium contributing to the granulation of platelets.

PRP incorporation in the carrier medium stimulates new tissue formation, as the granules of PRP will release relevant growth factors at ‘naturally determined’ rate, i.e. as the body needs.

The release rate of relevant growth factors can be accelerated in the presence of a local supply of calcium which is the result of either tricalcium phosphate incorporation in the shells or the use of calcium carbonate as a porogen during manufacture.

It is thus important that the bulking material particles must have a size in the range specified, ie a maximum outer dimension of 50 μm-250 μm so that they can be injected into a site requiring bulking. Larger particles will not be readily injectable for an endoscopic application. Typically an endoscopic needle would have a minimum gauge size of 23 and a laparoscopic needle a maximum gauge size of 16. Thus, it is not required that the particles of the invention have angiogenic potential (as is the case where particles are required for applications such as liver transplantations), i.e. they need not be of such a size so as to get formation of blood vessels inside the particles and which typically occurs when the particles are larger than 0.5 mm. Similarly, port openings of 20 μm-100 μm in accordance with the Invention permit tissue ingrowth into the insides of the particles, while larger port openings are required for blood vessel formation Inside the particles. Bulking material particles in accordance with the invention can be produced by the methods hereinbefore described. On the other hand, methods of making larger particles having for blood vessel formation, typically particles in the size range 0.5 mm-3 mm with ports larger than 100 μm, typically 200 μm or larger, are generally not suitable for making the smaller particles, having the smaller port sizes, in accordance with the invention.

1. A soft tissue bulking material, which includes a plurality of particles, with each particle comprising a rounded polymeric shell defining an internal cavity and having a maximum outer dimension of 50 μm-250 μm, and a single port or opening having a size or dimension that ranges from one-tenth of the particle's outer dimension up to the particle's outer dimension, in the shell, with the port or opening thus providing access to the cavity.

2. A bulking material according to claim 1, wherein the shells of the particles are substantially spherical so that their outer diameters are thus 50 μm-250 μm.

3. A bulking material according to claim 2, wherein the ports of the particles are substantially circular with their diameters thus ranging from one tenth of the diameters of the particles up to the respective diameters of the particles.

4. A bulking material according to claim 3, wherein the shells of at least some of the particles have micropores with dimensions <10 μm.

5. A bulking material according claim 4, wherein the shells of at least some of the particles have macropores with dimensions of from 10 μm to 50 μm.

6. A bulking material according to claim 5, wherein less than 20% of the outer surface areas of the shells of the particles are occupied by macropores.

7. A bulking material according to claim 6, wherein the shell includes, as a composite with the polymer or absorbed or otherwise attached thereto, at least one additive selected from a calcium phosphate compound, a contrast agent, a therapeutic agent, a growth factor, autologous platelet rich plasma, normal human cells, and autologous stem cells.

8. A bulking material according to claim 7, wherein the shells of at least some of the particles have an outer surface layer of hydroxyapatite and/or tricalcium phosphate.

9. An injectable soft tissue bulking composition, which includes
(i) a soft tissue bulking material as claimed in claim 8; and
(ii) a compatible carrier medium in which the bulking material particles are suspended, with the composition having a consistency that renders it suitable for application by injecting it into soft tissue.

10. A composition according to claim 9, wherein the carrier medium includes a carrier medium material selected from collagen, chitosan, alginate, polyvinyl pyrrolidone, silicone oil, gelatin, fat, hyaluronic acid, saline, water, plasma, aqueous solution, glycols, medium chain triglycerides, glycerides, glycerol, B-glucan & agarose solution, ethyl lactate, hydroxypropyl methylcellulose, poloxamers or poly (N-isopropylacrylamide) or a derivative thereof, dissolved in a solvent.

11. A composition according to claim 10, wherein the carrier medium is in liquid form at room temperature and is adapted to undergo a phase change to gel form when injected into human soft tissue.

12. A composition according to claim 11, wherein the carrier material medium comprises highly purified glutaraldehyde crosslinked bovine dermal collagen.

13. A composition according to claim 10, wherein the carrier medium is a pseudoplastic liquid, thus providing reduced viscosity within the relatively high shear environment during injection, but higher viscosity for stabilising or suspending the injected material after injection.

14. A composition according to claim 13, wherein the carrier medium comprises hyaluronic acid dissolved in water.

15. A composition according to claim 14, wherein the carrier medium includes, as an additive, a contrast agent, autologous platelet rich plasma, normal human cells, and/or autologous stem cells.
